(19) United States
(12) Patent Application Publication
(10) Pub. No.: US 2006/0239919 A1
(43) Pub. Date: Oct. 26, 2006

(54) MR coronary angiography with a fluorinated nanoparticle contrast agent at 1.5 T

(76) Inventors: Samuel A. Wickline, St. Louis, MO (US); Gregory M. Lanza, St. Louis, MO (US)

Correspondence Address: THOMPSON COBURN, LLP, ONE US BANK PLAZA, SUITE 3500, ST LOUIS, MO, 63101, US

(21) Appl. No.: 11/367,971

(22) Filed: Mar. 3, 2006

Related U.S. Application Data
(60) Provisional application No. 60/658,460, filed on Mar. 4, 2005.

(51) Int. Cl. A61B 5/055

(52) U.S. Cl. 424/9.3

(57) ABSTRACT

Disclosed herein is a medical imaging technique that uses a fluorinated nanoparticle contrast agent for imaging of an interior portion of a body. The fluorinated nanoparticles preferably comprise nontargeted intravascular fluorocarbon or perfluorocarbon nanoparticles. The interior body portion may be a patient's vasculature, and the medical imaging is preferably noninvasive MR angiography, which may encompass (either for 2D imaging or 3D imaging) MR coronary angiography, MR carotid angiography, MR peripheral angiography, MR cerebral angiography, MR arterial angiography, and MR venous angiography. Coils tuned to match to the ¹⁹F signal can be used, or dual tuned coils for ¹⁹F and ¹H imaging can be used. Clinical field strengths (e.g. 1.5 T) and clinical doses may be used while still providing effective images.

### CROSS-REFERENCE AND PRIORITY CLAIM TO RELATED APPLICATION

This application claims priority to U.S. provisional patent application 60/658,460 filed Mar. 4, 2005 and entitled “MR Coronary Angiography with a Fluorinated Nanoparticle Contrast Agent at 1.5 T”, the entire disclosure of which is incorporated by reference herein.

### FIELD OF THE INVENTION

The present invention is generally directed to the field of medical imaging with fluorinated contrast agents, particularly ¹⁹F magnetic resonance (MR) imaging of a vasculature with fluorinated nanoparticle contrast agents at clinical field strengths.

### BACKGROUND AND SUMMARY OF THE INVENTION

Contrast-enhanced coronary artery angiography with magnetic resonance imaging (MRI) provides a potentially attractive alternative to X-ray angiography for visualization of coronary artery disease because it is noninvasive and does not employ ionizing radiation. However, both the sensitivity and specificity of this technique have yet to meet the expectations required for clinical adoption.

In an effort to provide alternative and improved techniques for angiography, the inventors have developed a contrast agent for use with MRI that does not depend on detection of the conventional proton signal, but instead utilizes the unique signal from fluorine species contained within a nanoparticulate emulsion. Because ¹⁹F can generate a measurable signal for MRI without any perceptible background tissue signal, the inventors sought to evaluate this contrast agent's performance for possible use in coronary artery angiography. The low natural abundance of ¹⁹F in physiological tissues, however, often necessitates the use of high magnetic field strengths and/or long scan times. The high concentration of fluorine in the agent of the present invention makes it practical to rapidly image small vessels at clinical field strengths (1.5 T). In the description set forth below, the inventors demonstrate “proof of principle” by using this contrast agent to image the coronary arteries of an ex vivo pig heart as well as the carotid arteries of a living rabbit.

While the use of fluorine contrast agents for MRI is not a new concept, conventional ¹⁹F MRI techniques have presented significant hurdles for clinical applications. First, many of the fluorinated contrast agents in use have a complicated ¹⁹F NMR frequency spectrum due to the presence of molecularly inequivalent fluorine atoms in the structure. Compared to ¹H NMR, ¹⁹F manifests larger chemical shifts such that the peak splitting caused by the inequivalent fluorine atoms is quite large and not easily recombined into a single signal. As frequency is used as an indication of position in MRI, this translates into “ghosting” of the image and inaccurate positioning for slice selection. Methods for overcoming this problem include narrow-bandwidth excitation, which can cause loss of available signal, or deconvolution, which frequently amplifies noise. The preferred fluorinated contrast agent used in the present invention, perfluoro-15-crown-5 ether, is unique in that all of its fluorine atoms are chemically equivalent, so that all 20 atoms contribute to the image signal without the requirement for special deconvolution strategies. Furthermore, to overcome the inherently low signal available with fluorine MRI, known practices used some combination of high field strengths, large doses (˜50% of blood volume replaced), and/or long scan times, all of which compromise applications in clinical imaging.

In an effort to fill this need in the art, the inventors herein have developed a ¹⁹F-based intravascular contrast agent that could improve contrast-enhanced MRI coronary angiography by allowing spatially matched detection of two different MR signals, ¹⁹F and the standard ¹H. This intravascular nanoparticle emulsion offers a unique spectral signature with no background signal because of the absence of detectable fluorine elsewhere in the body. The inventors herein disclose that performance of contrast-enhanced MRI coronary angiography in accordance with the present invention can be improved through proper selection of a fluorine contrast agent (preferably a perfluorocarbon with 20 equivalent fluorine molecules), appropriate selection and use of RF coils, and appropriate selection of an MRI technique such as an efficient steady-state free procession sequence.

Accordingly, disclosed herein is a method comprising: using a nontargeted intravascular fluorinated nanoparticle contrast agent for medical imaging of an interior portion of a body. The fluorinated nanoparticles preferably comprise fluorocarbon or perfluorocarbon nanoparticles. The interior body portion may be a patient's vasculature, and the medical imaging is preferably noninvasive MR angiography, which may encompass (either for 2D imaging or 3D imaging) MR coronary angiography, MR carotid angiography, MR peripheral angiography, MR cerebral angiography, MR arterial angiography, and MR venous angiography. The measurement technique for the MR angiography may comprise any selected from the group consisting of steady state free precession imaging, routine gradient echo imaging, spin echo imaging, echo planar imaging, and projection imaging, among other standard methods.

The preferred intravascular perfluorocarbon nanoparticle contrast agent, which remains intravascular while circulating within the bloodstream of the patient, may comprise a plurality of perfluorocarbon nanoparticles, each perfluorocarbon nanoparticle having a diameter in a range of about 200 nm to about 300 nm. These perfluorocarbon nanoparticles can be made by emulsification and are preferably surrounded by a lipid surfactant monolayer. Furthermore, these perfluorocarbon nanoparticles are preferably not targeted with any binding ligands so that the agent is not removed from the circulation by targeted binding. Gd chelates may be present on the contrast agent's surface to produce a signal detectable with proton imaging. Moreover, the contrast agent may comprise a mixture that includes a high concentration of fluorine, such as a mixture that comprises approximately 98% perfluorocarbon nanoparticles. The perfluorocarbon nanoparticles can be liquid at body temperature, less than approximately 5% gas at body temperature, or gaseous at body temperature.

Coils tuned to match to the ¹⁹F signal can be used, or dual tuned coils for ¹⁹F and ¹H imaging can be used. Suitable field strengths for MR imaging with the inventive technique include 1.5 T, 3 T, 7 T, and 11.7 T. Furthermore, it is believed that field strengths greater than 7 T could be used in patients. Spectral peak saturation techniques can be used to reduce the signal from unwanted peaks present in certain perfluorocarbon components for imaging so that signal localization can be achieved by avoiding chemical shifts.

Among the applications of the present invention in connection with ¹⁹F-based contrast agents for contrast-enhanced MRI coronary (or carotid, peripheral, cerebral, or other arterial or venous angiography) angiography are (1) interventional: injection of the agent into the artery with first pass detection of the bolus passing through a field of interest, (2) intravenous injection of the agent with first pass imaging, and (3) intravenous injection of the agent with “steady-state”, “quasi-steady-state”, or time-delayed imaging after sufficient build-up of agent concentration in the bloodstream to give a detectable signal from vasculature (e.g., from 10 minutes to 1-2 hours after iv injection).

According to one embodiment of the invention, this inventive technique allows for the performance of spatially matched detection of different MR signals involving ¹⁹F and ¹H. The nanoparticle emulsion can include Gd chelates on its surface, and the ¹H signal can be imaged from these Gd chelates, and the ¹⁹F signal can be imaged from the core fluorocarbon (FC) or perfluorocarbon (PFC) nanoparticles. Interleaved MRI acquisitions can be used to allow spatial registration.

According to another embodiment of the invention, this inventive technique allows for the reduction or elimination of background tissue signal in MR imaging using ¹⁹F. Further still, venous blood can be separated from arterial blood based on the differential signal from F due to the changes in oxygen concentrations between veins and arteries, and from the effects on relaxation times of ¹⁹F under high and low oxygen tension.

According to yet another embodiment of the invention, this inventive technique allows for spectroscopic delineation of the concentration of ¹⁹F in the blood pool or vascular space. Different ¹⁹F species could be detected (or imaged) with the ability to separate different spectral peaks of the various FC or PFC compounds used to create the nanoparticles.

According to yet another aspect of the invention, this inventive technique can be applied to image the GI tract, either upper or lower. Further still, this inventive technique can be applied to cystourethrography to image the bladder and/or urethra.

Additional background information regarding the field of the invention can be found in the following references, the entire disclosures of each of which are incorporated herein by reference: Danias P G, Roussakis A, Ioannidis J P., Diagnostic performance of coronary magnetic resonance angiography as compared against conventional X-ray angiography: a meta-analysis, J Am Coll Cardiol 2004; 44(9): 1867-76; Flacke S, Fisher S, Scott M J, et al., Novel MRI contrast agent for molecular imaging of fibrin: implications for detecting vulnerable plaques, Circulation 2001. 104(11):1280-1285; Dardinski B J, Sotak C H., Rapid tissue oxygen tension mapping using 19F inversion-recovery echo-planar imaging of perfluoro-15-crown-5-ether, Magen Reson Med 1994. 32(1):88-97; and Mason R P, Hunyan S, Le D, et al., Regional tumor oxygen tension: fluorine echo planar imaging of hexafluorobenzene reveals heterogeneity of dynamics, Int J Radiat Oncol Biol Phys 1998: 42(4):747-50; U.S. patents U.S. Pat. Nos. 5,989,520 and 6,821,506; and U.S. patent application publications 2002/0102216A1, 2002/0168320A1, 2003/0129136A1, 2003/0185760A1, 2003/0215392A1, and 2004/0115192A1.

These and other aspects of the present invention will be in part apparent and in part pointed out to those having ordinary skill in the art following the teachings herein.

### BRIEF DESCRIPTION OF THE DRAWINGS

FIG. 1 shows a series of time-elapsed ¹⁹F images acquired during a phantom imaging experiment;

FIGS. 2(a)-(d) depict time-elapsed ¹⁹F images acquired during injection of fluorinated nanoparticles into the left coronary artery of an excised pig heart;

FIG. 2(e) depicts a ¹H image (single coronal slice through left ventricle, labeled LV) corresponding to the images of FIGS. 2(a)-(d);

FIG. 3(a) is a single slice ¹H image through a rabbit neck;

FIG. 3(b) is a ¹⁹F projection image corresponding to the image of FIG. 3(a) that was acquired during nanoparticle injection;

FIG. 3(c) is a false color overlay of ¹⁹F image (arrow) of FIG. 3(b) onto the ¹H image of FIG. 3(a) showing the anatomic location of the ¹⁹F signal;

FIG. 4 shows a series of time-elapsed images acquired during in vivo experiment B;

FIG. 5 shows a series of time-elapsed images acquired during in vivo experiment C; and

FIG. 6 is a graph showing a correlation between the dose of fluorine administered to rabbits and the resulting blood concentration used for the steady-state imaging experiment.

### DETAILED DESCRIPTION OF THE PREFERRED EMBODIMENT

The following describes a methodology for practicing an embodiment of the present invention.

To enable ¹⁹F imaging on the inventors' clinical 1.5 T Philips MR scanner (an NT Intera available from Philips Medical Systems of Andover, Mass.), the system was modified to include a specialized channel tuned for fluorine nuclei, and a series of surface and volume RF coils tuned to the same frequency (60.1 MHz) were developed. These coils were used for both transmission and receive of the MR signal. A 13.5 cm diameter and 14.5 cm long saddle coil was designed for homogeneous transmission using copper foil formed onto a plexiglass frame. High-voltage variable capacitors made of Teflon for MR compatibility (available from Johanson of Boonton, N.J. and from Voltronics, of Denville, N.J.) were used for tuning and matching to different loads, and a balun network was added for improved isolation. To increase the sensitivity for in vivo imaging, a 7 cm square surface coil was created by chemical etching of copper-clad glass epoxy. Variable tuning and matching capacitors were used to accommodate different loads, and splitting of the matching capacitance provided adequate isolation.

However it should be noted that other RF coils can be used in the practice of the present invention. For example, the inventors envision that the use of a quadrature birdcage coil can be advantageous. Further, the inventors envision that the use of different coils for transmission and reception can be advantageous—for example, the use of a homogeneous volume coil (e.g., a single turn solenoid) for transmission and a surface coil for reception.

It should also be noted that for MR scanners having fluorine channels and appropriate associated coils, the specialized channel and coils are not needed. For example, scanners with multinuclear imaging capabilities are available from manufacturers such as Philips, G E, and Siemens. The inventors herein further believe that any of a number of known tuned coils for fluorine imaging can be used in the practice of the present invention.

A preferred fluorinated contrast agent for use with the present invention is a perfluorocarbon nanoparticle emulsion. Perfluorocarbon nanoparticles (20% v/v perfluoro-15-crown-5-ether; ˜250 nm diameter, 18.2 M fluorine concentration) were formulated by microemulsification for the MR angiography experiments as described in Flacke et al., Novel MRI contrast agent for molecular imaging of fibrin: implications for detecting vulnerable plaques, Circulation 2001;104:1280-1285; and Lanza et al., Targeted antiproliferative drug delivery to vascular smooth muscle cells with an MRI nanoparticle contrast agent: Implications for rational therapy of restenosis, Circulation 2002;106:2842-2847, the entire disclosures of both of which are incorporated herein by reference.

These nanoparticle emulsions were composed of 20% (v/v) of perfluoro-15-crown-5 ether (C₁₀F₂₀O₅; available from the Exfluor Research Corp. of Round Rock, Tex.), 2% (w/v) safflower oil, 2% (w/v) of a surfactant co-mixture, and 1.7% (w/v) glycerin, with water comprising the balance. The surfactant co-mixture was comprised of 30 mol % lipophilic gadolinium-diethylene-triamine-pentaacetic acid-bis-oleate (Gd-DTPA-BOA; available from Gateway Chemical Technologies of St. Louis, Mo.), 60 mol % lecithin (available from Avanti Polar Lipids, Inc. of Alabaster, Ala.), 8 mol % cholesterol (available from Sigma Chemical Co. of St. Louis, Mo.), and 2 mol % dipalmitoyl-phosphatidylethanolamine (available from Avanti Polar Lipids, Inc. of Alabaster, Ala.). Particle sizes were determined at 25° C. with a laser light scattering submicron particle sizer (available from Malvern Instruments of Malvern, Worcestershire, UK). Perfluoro-15-crown-5 ether (CE) is a cyclic perfluorocarbon with 20 equivalent fluorine atoms per molecule.

It should be noted that the inventors believe that any of several FC or PFC nanoparticle emulsions may be used in the practice of the present invention, examples of which are disclosed in the following U.S. patents and U.S. published patent applications: U.S. Pat. Nos. 5,690,907, 5,780,010, 5,958,371, 6,548,046, 6,676,963, 2003/0086867A1, 2004/0058951A1, and 2004/0248856A1, the entire disclosures of each of which are incorporated herein by reference.

Phantom imaging: Flexible plastic extension tubing (available from Baxter Healthcare Corp of Deerfield, Ill.) was formed into a loop and placed inside of the saddle coil described above between saline IV bags to minimize susceptibility artifact. Undiluted CE nanoparticles were slowly injected into the tubing, and the ¹⁹F signal was imaged using a dynamic steady-state free procession imaging sequence (balanced FFE (bFFE) sequence, 4 ms TR, 1.4 ms TE, 320 mm FOV, 2.5×2.0×73 mm reconstructed resolution, 4 signal averages, 90° flip angle, 1.3 s/dynamic). ¹H multislice images were also acquired for colocalization of the ¹⁹F signal using a built-in quadrature body coil (turbo spin echo sequence with turbo factor of 22, 5 slices, 1518 ms TR, 150 ms TE, 320 mm FOV, 1.25×1.01×6 mm resolution, 6 signal averages).

Ex vivo experiment: Crown ether nanoparticles were slowly hand-injected through a 2 F diameter balloon catheter into the left main coronary artery of an isolated and heparinized pig heart.

In vivo experiment A: A 3 F balloon catheter was inserted into the femoral artery of an anesthetized New Zealand white rabbit and advanced to the right carotid artery. Nanoparticles were injected slowly and continuously into the flowing artery during scanning, up to a total volume of ˜7 cc per injection.

For the ex vivo experiment and in vivo experiment A, a series of dynamic “balanced” FFE ¹⁹F projection scans (TR=4 ms, TE=1.5 ms, matrix=2×2.5×70 mm, 2.8 s per dynamic) were acquired using a 13 cm transmit and receive Helmholtz (ex vivo) or a 10 cm transmit and receive surface coil (in vivo). Corresponding projection and multi-slice ¹H images of the anatomy were also acquired.

In vivo experiments B and C: Male New Zealand white rabbits (n=4) were anesthetized using an intramuscular injection of ketamine (35 mg/kg) and xylazine (7 mg/kg) followed by maintenance using IV delivery of a ketamine and xylazine (2 mg/kg/min and 1 mg/kg/min respectively) mixture. These rabbits were intubated and maintained on 2 L/min 100% O₂ for the duration of the exam. For first pass imaging, two of the rabbits were catheterized using a femoral artery cutdown technique under sterile conditions. A 4 F Fogarty catheter (available from Edwards Lifesciences of Irvine, Calif.) was then advanced to the left carotid artery under fluoroscopy guidance. The animals were then positioned in the MR scanner for imaging, and ¹H surveys and time-of-flight angiography scans of the neck region were acquired using a quadrature body coil for transmission and a 4 cm surface coil for receive (multiple 2D inflow FFE sequence, 160 mm FOV, 2.8 ms TE, 6.8 ms TR, 4 signal averages, 40 slices, 0.31×0.31×4 mm reconstructed resolution, 60° flip angle, 2 min:19 s scan time). CE nanoparticles were injected (1-2 ml) into the vessel, during which ¹⁹F dynamic projection images were acquired with a 7 cm surface coil (bFFE sequence, 260 mm FOV, 1.7 ms TE, 3.5 ms TR, 512 signal averages, 2.03×2.03×50 mm reconstructed resolution, 90° flip angle, ˜2 min scan time).

In two different rabbits, steady state intravascular fluorine concentrations were produced for angiographic imaging by administering up to five sequential intravenous 0.5 mL/kg doses of crown ether nanoparticles at approximately 20 minute intervals. Subsequent to each injection, ¹⁹F projection images of the vessels in the neck were acquired (bFFE sequence, 260 mm FOV, 1.4 ms TE, 4 ms TR, 512 signal averages, 2.03×2.03×20 mm reconstructed resolution, 60° flip angle, ˜2 min scan time). An intravenous blood sample (1 ml) was removed and analyzed for gadolinium content after image acquisition, which allowed measurement of the nanoparticle concentration in blood using gadolinium as the “tracer” (see below). In total, five doses were delivered to the first rabbit, while six were delivered to the second. Total imaging time was approximately 2 hours.

Analysis of blood samples for fluorine content: To relate the ¹⁹F signal intensity to concentration of fluorine, blood samples from each rabbit used for “steady-state” angiography were analyzed for gadolinium content using both relaxation time measurements and neutron activation analysis. Essentially, the relationship between the gadolinium and fluorine content in the nanoparticles is established in the formulation process, so that one can be calculated from the other. The gadolinium was included in the particle formulation purely as a method for determining the fluorine concentration. A benchtop spectrometer (a MiniSpec spectrometer available from Bruker Optics of Billerica, Mass.) at 0.47 T was used for relaxation time measurements. A calibration curve was obtained by doping blood from an untreated rabbit with known volumes of the crown ether, gadolinium-containing, nanoparticles. Six amounts, ranging from 0 to 20 □L of emulsion, were added to 0.5 mL of blood, producing fluorine concentrations of 0 to 0.49 M. An inversion recovery pulse sequence was used with 10 inversion delay times that varied according to the concentration of gadolinium present. A minimum of three T₁ measurements was averaged for each sample, and measurements were made at 40° C.

Four of the six calibration samples were also prepared for neutron activation analysis for absolute quantification of the gadolinium content at the Research Reactor facility at the University of Missouri (MURR). See Landsberger S., Delayed instrumental neutron activation analysis. In: Alfassi Z B, editor. Chemical Analysis by Nuclear Methods. New York: John Wiley & Sons; 1994. p 122-140, the entire disclosure of which is incorporated herein by reference. After lyophilization of 50 □L of each blood sample, the mass of gadolinium was calculated from the beta decay of ¹⁶¹Gd produced through neutron capture on ¹⁶⁰Gd. Individual samples and standards were irradiated in a thermal neutron flux of about 5×10¹³ n·cm⁻²·s⁻¹ for 7 seconds, allowed to decay for 30 seconds, and counted on a high-resolution gamma-ray spectrometer for 300 seconds. This method provided an exact measure of the gadolinium content in each sample, which allowed precise calculation of the relaxivity of the nanoparticles at 0.47 T.

Blood samples from the rabbits injected with nanoparticles were analyzed in a similar manner with both relaxation measurements and neutron activation for total gadolinium content. The relaxivity determination using the calibration samples allowed calculation of the absolute concentration of gadolinium in the blood samples, and neutron activation was used to verify this measurement.

As a result of these experiments, the following results were achieved.

The undiluted nanoparticles, as formulated, contain 12.14 M fluorine atoms (or alternatively, 0.61 M perfluoro-15-crown-5 ether) and approximately 40,000 gadolinium atoms per particle. The nominal particle diameter was 185 nm. The longitudinal relaxivity of the particles was 12.3 s⁻¹ mM⁻¹ in rabbit blood at 40° C. and 0.47 T, when expressed in terms of the concentration of gadolinium chelates (regression line: R₁(1/s)=12.3·[Gd](mM)+0.90; r²>0.99).

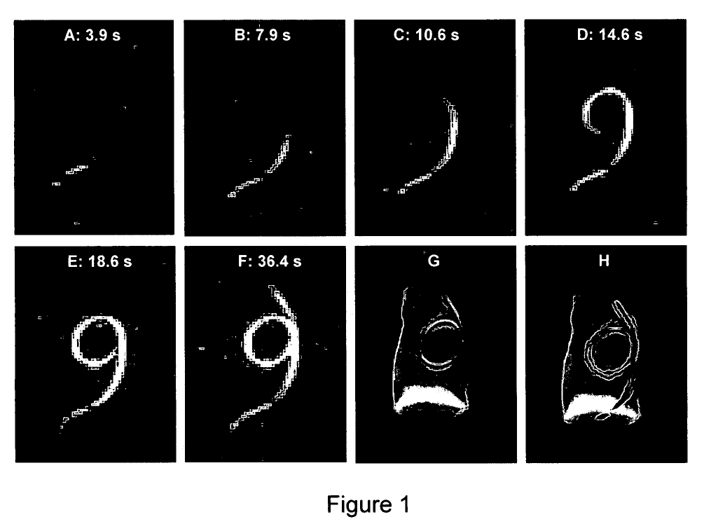

FIG. 1 (panels A-F) shows fluorine images for the phantom imaging experiment after injection of undiluted nanoparticles through 1.9 mm diameter tubing using a dynamic bFFE sequence. The time of acquisition after the injection started is labeled on each panel in seconds. Only selected images from the series are shown to demonstrate the movement of the particles through the tubing. This resulted in a signal-to-noise ratio of approximately 14, which is equivalent to the contrast-to-noise ratio because of the lack of competing background signal. Note, from panels G and F in FIG. 1, that the fluorine signal overlays precisely with the tubing in the ¹H image, indicating that the frequency shift does not result in localization problems. See also panel H which is a false color overlay of panel F onto panel G showing the colocalization of the ¹⁹F signal with the tubing.

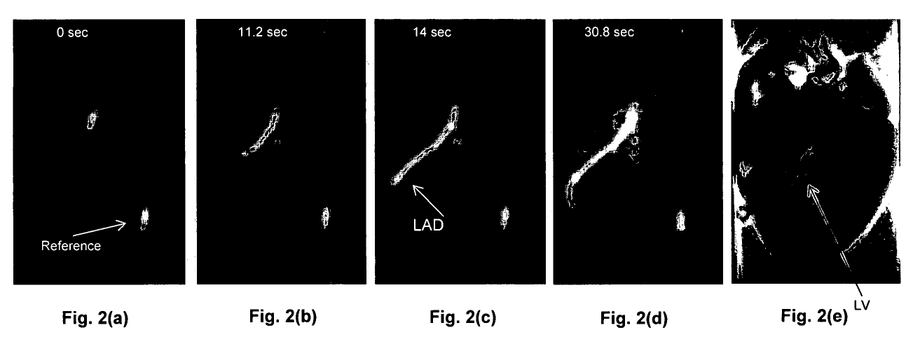

FIGS. 2(a)-(e) depict the left coronary artery tree of the ex vivo pig heart as seen with ¹⁹F MRI during injection of the nanoparticles as per the ex vivo experiment. FIGS. 2(a)-(d) are time-elapsed ¹⁹F images acquired during injection of fluorinated nanoparticles into the left coronary artery of the excised pig heart. FIG. 2(e) shows the corresponding ¹H image (single coronal slice through left ventricle, labeled LV). This technique generated a signal to noise ratio (SNR) of 19.7 from the vessel with a scan time of only 2.8 seconds per image. Due to the lack of background signal present in these images, the contrast-to-noise ratio (CNR) equals SNR-1, or ˜19, which is quite high for a relatively unoptimized imaging procedure.

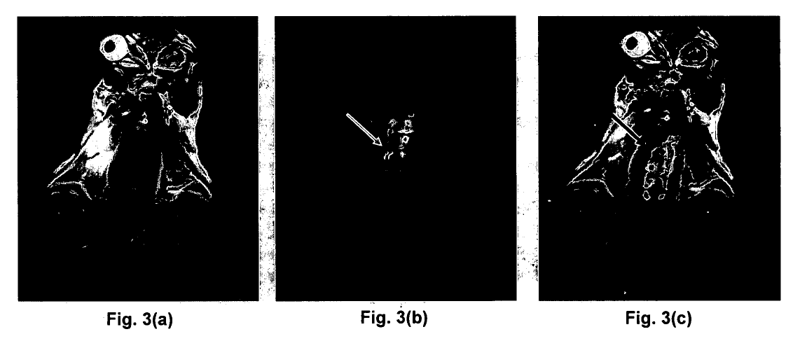

FIGS. 3(a)-(c) show the results of the in vivo imaging of the rabbit carotid arteries as per in vivo experiment A. FIG. 3(a) depicts a single slice ¹H image through a rabbit neck. FIG. 3(b) depicts a ¹⁹F projection image acquired during nanoparticle injection. FIG. 3(c) depicts a false color overlay of ¹⁹F image (arrow) onto ¹H image showing the anatomic location of the ¹⁹F signal. As can be seen, the inventors were able to visualize the vasculature (both arteries and veins) dynamically with this scanning technique. In this case, the SNR=10.7 (CNR=9.7), which was lower than the ex vivo, likely due to dilution effects caused by flowing blood.

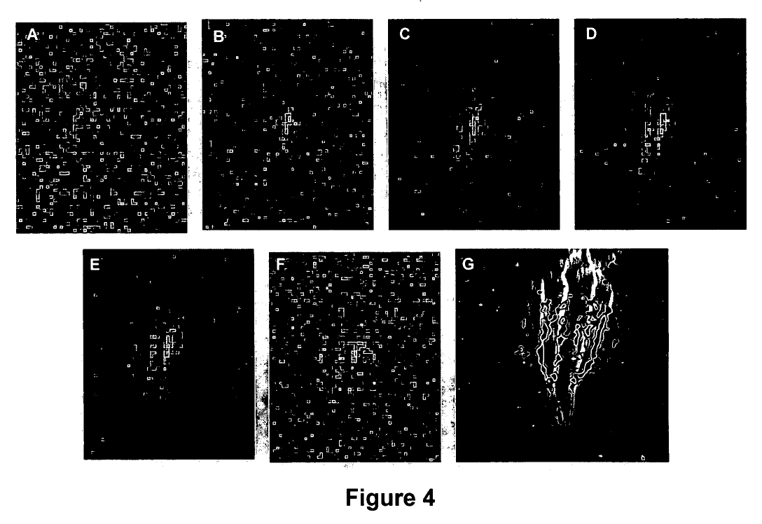

The first set of images for in vivo experiment B as shown in FIG. 4 was acquired by placing the catheter in the carotid artery to permit delivery of high local concentrations of nanoparticles. Panels A-F of FIG. 4 show Dynamic ¹⁹F images of crown ether (CE) nanoparticles injected via a catheter into the left carotid artery of a live rabbit. Panel A of FIG. 4 shows the first two injections were not sufficient to generate detectable ¹⁹F signal after being diluted in the total rabbit blood volume. Panels B-E of FIG. 4 show the accumulation of signal during the injection. Panel F of FIG. 4 shows the washout of the signal after the injection ceased. Panel G of FIG. 4 shows an overlay of the ¹⁹F signal from a longer scan onto a MIP of a time of flight angiography scan. Note the co-registration of the ¹⁹F signal with the vessels in the neck as shown in panel G.

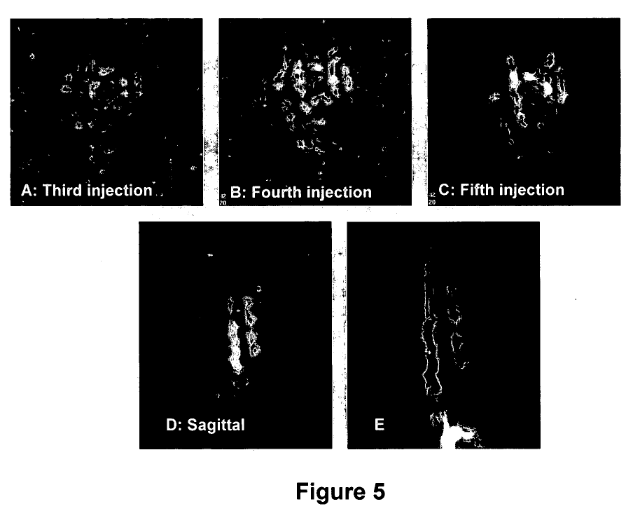

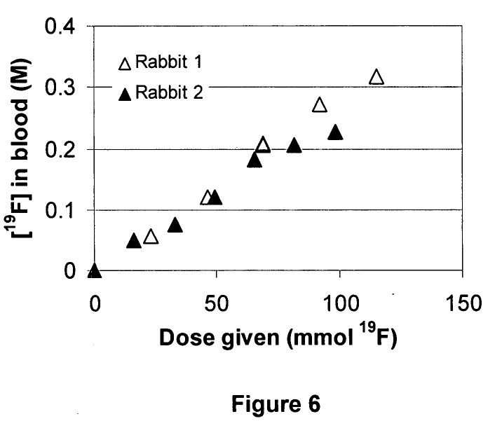

As a final step in evaluating this method for ¹⁹F angiography, we injected nanoparticles intravenously to determine the minimum dosage required for visualization of the neck vasculature as per in vivo experiment C (see FIG. 5). Each 0.5 mL/kg injection increased the systemic concentration of ¹⁹F in the rabbit's blood in a predictable manner (see FIG. 6), due in part to the intravascular retention and long circulating half-life of these particles. See Flaim S F, Pharmacokinetics and side effects of perfluorocarbon-based blood substitutes, Artif Cells Blood Substit Immobil Biotechnol 1994;22(4):1043-1054, the entire disclosure of which is incorporated herein by reference. FIG. 6 shows a correlation between the dose of fluorine administered to rabbits and the resulting blood concentration used for the steady-state imaging experiment. The concentration of fluorine in the blood was determined by measuring the concentration of gadolinium and using the known ratio of gadolinium to crown ether in the emulsion to calculate fluorine concentration. Note that rabbit 2 exhibited a smaller increase in blood concentration as a function of dose at the higher doses.

Panels A-C of FIG. 5 show ¹⁹F coronal projections through the first steady-state injection rabbit acquired after each injection. Panel D of FIG. 5 shows a ¹⁹F sagittal projection through the neck of the second rabbit after the sixth dose of particles. Panel E of FIG. 5 shows an overlay of the ¹⁹F image in blue onto a sagittal MIP of a ¹H time of flight angiography scan in the same rabbit.

For the first rabbit, a minimum of 3 doses (1.5 mL/kg) was required to detect the ¹⁹F signal (panel A of FIG. 5). Each subsequent dose increased the signal, and provided better images of the vessels (panels B-C of FIG. 5). The second rabbit required a higher number of doses (6) for the same signal level obtained for the first rabbit (panels D-E of FIG. 5). The blood testing indicated that this rabbit experienced a smaller increase in serum ¹⁹F for each dose as compared to the first rabbit (FIG. 6), likely a function of the different distribution volumes in each rabbit. However, a ¹⁹F sagittal projection image of the neck did allow visualization of two vessels (panel E of FIG. 5), which matched those observed in a MIP of a PCA scan in the same orientation.

This work demonstrates the possibility of using intravascular perfluorocarbon nanoparticle contrast agents for MR angiography of vasculature that is similar in size to a human coronary artery with ¹⁹F MRI. To the inventors knowledge, this is the first demonstration of imaging of small vessels at 1.5 T with sufficient temporal resolution to view the first pass injection through a catheter. Furthermore, these results indicate the ability to image the steady-state blood signal from the nanoparticles at moderate doses, corresponding to 1.5-2.5 mL emulsion per kg body weight (or, equivalently, 0.5-0.9 g perfluorocarbon per kg). This dosage is well within the “absolute no effect dose” of 2.7-9 g PFC/kg determined using other PFC emulsions, which indicates that it should be safe for use in patients.

To date, perfluorocarbon contrast agents and hyperpolarized gases have been the only intravascular agents developed for MRI that can be used to generate images of the vasculature with no background signal from surrounding tissues. See Moller et al., Magnetic resonance angiography with hyperpolarized 129Xe dissolved in a lipid emulsion, Magn Reson Med 1999;41(5):1058-1064, the entire disclosure of which is incorporated herein by reference. The imaging methods described herein may ultimately allow estimation of lumen diameter in much the same way that traditional angiography is used. ¹H imaging, while also successful in this regard, requires the use of special imaging techniques or contrast agent administration in order to obtain sufficient signal from the blood in the vessels. Alternatively, perfluorocarbon nanoparticles might provide an unambiguous signal from the vessel lumen under steady state imaging conditions. While MR techniques using hyperpolarized gases dissolved in lipids also show these same benefits, the perfluorocarbon particles do not require expensive specialized machinery for production and can be used “off-the-shelf.” Furthermore, hyperpolarized gases cannot be used under steady state imaging conditions since the signal dissipates rapidly after injection due to fast relaxation.

Furthermore, contrary to known practices which require some combination of high field strengths and large doses, the current demonstration of fluorine angiography utilized far smaller doses of nanoparticles that would be practical for clinical application, especially under conditions of steady state imaging. The high level of signal was obtained with the use of a “balanced” gradient echo imaging technique, which allows for rapid scanning and higher signal levels than obtained with any other sequence to date. See Scheffler et al., Principles and applications of balanced SSFP techniques, Eur Radiol 2003;13(11):2409-2418, the entire disclosure of which is incorporated herein by reference. By fully compensating for the dephasing effects of the read-out gradient, this pulse sequence is able to refocus “left-over” magnetization after the end of a pulse train, unlike other common sequences. In addition, the maximum signal obtained occurs when the sample of interest manifests comparable T₁ and T₂ times. Perfluoro-15-crown-5 ether is characterized by a very high T₂ relaxation time at 1.5 T, which renders this sequences particularly suitable for angiography. The surprising amount of signal observed at 1.5 T with only modest amounts of fluorinated nanoparticles delivered intravenously lends credibility to the prospect for noninvasive fluorine angiography, particularly considering the use of conventional imaging methods. Further optimization likely will improve the image quality and appearance.

Potential issues with translating this approach to in vivo coronary imaging may include loss of signal due to heart motion, partial volume effects, and possible oxygenation-mediated changes in perfluorocarbon signal. However, incorporation of cardiac gating together with sequence optimization should mitigate these limitations in part. The surprisingly high level of contrast generated by this contrast agent in these experiments offers the potential for peripheral injection of nanoparticles for non-invasive MR angiography of the coronary arteries with no competing background signal and potential for spatially matched anatomical images.

While the present invention has been described above in relation to its preferred embodiment, various modifications may be made thereto that still fall within the invention's scope, as would be recognized by those of ordinary skill in the art. Such modifications to the invention will be recognizable upon review of the teachings herein. As such, the full scope of the present invention is to be defined solely by the appended claims and their legal equivalents.

What is claimed is:

1. A method comprising:
using a nontargeted intravascular fluorinated nanoparticle contrast agent for medical imaging of an interior portion of a body.

2. The method of claim 1 wherein the using step comprises using a nontargeted intravascular fluorocarbon nanoparticle contrast agent or a nontargeted intravascular perfluorocarbon nanoparticle contrast agent as the contrast agent for the medical imaging.

3. The method of claim 2 wherein the using step comprises:
using the nontargeted intravascular fluorocarbon or perfluorocarbon nanoparticle contrast agent for medical imaging of a vasculature.

4. The method of claim 3 wherein the using step further comprises:
using the nontargeted intravascular perfluorocarbon nanoparticle contrast agent for medical imaging of the vasculature.

5. The method of claim 4 wherein the medical imaging comprises angiography.

6. The method of claim 5 wherein the angiography comprises MR angiography.

7. The method of claim 6 wherein the MR angiography is noninvasive MR angiography.

8. The method of claim 6 wherein the MR angiography comprises ¹⁹F MRI.

9. The method of claim 8 wherein a measurement technique for the MR angiography comprises at least one selected from the group consisting of steady state free precession imaging, routine gradient echo imaging, spin echo imaging, echo planar imaging, and projection imaging.

10. The method of claim 8 wherein the intravascular perfluorocarbon nanoparticle contrast agent comprises a plurality of perfluorocarbon nanoparticles, each perfluorocarbon nanoparticle having a diameter in a range of about 200 nm to about 300 nm.

11. The method of claim 10 wherein the perfluorocarbon nanoparticles are made by emulsification and are surrounded by a lipid surfactant monolayer.

12. The method of claim 11 wherein the contrast agent remains intravascular while circulating within the bloodstream of the patient.

13. The method of claim 11 wherein the perfluorocarbon nanoparticles are not targeted with any binding ligands.

14. The method of claim 13 wherein the contrast agent comprises a high concentration of fluorine.

15. The method of claim 14 wherein the contrast agent comprises a mixture, the mixture being comprised of approximately 98% perfluorocarbon nanoparticles.

16. The method of claim 13 wherein the perfluorocarbon nanoparticles are liquid at body temperature.

17. The method of claim 13 wherein the perfluorocarbon nanoparticles are less than approximately 5% gas at body temperature.

18. The method of claim 13 wherein the perfluorocarbon nanoparticles are gaseous at body temperature.

19. The method of claim 13 wherein the MR angiography comprises MR coronary angiography.

20. The method of claim 13 wherein the MR angiography comprises MR carotid angiography.

21. The method of claim 13 wherein the MR angiography comprises MR peripheral angiography.

22. The method of claim 13 wherein the MR angiography comprises MR cerebral angiography.

23. The method of claim 13 wherein the MR angiography comprises MR arterial angiography.

24. The method of claim 13 wherein the MR angiography comprises MR venous angiography.

25. The method of claim 13 wherein the MR angiography comprises 2D MR angiography.

26. The method of claim 24 wherein the MR angiography comprises 3D MR angiography.

27. The method of claim 13 wherein the using step comprises:
intravascularly injecting the contrast agent into the vasculature.

28. The method of claim 27 wherein the injecting step comprises intravascularly injecting the contrast agent into an artery, the method further comprising:
performing MR angiography on the vasculature with first pass detection of a bolus passing through a field of interest.

29. The method of claim 27 wherein the injecting step comprises intravenously injecting the contrast agent into an artery, the method further comprising:
performing MR angiography on the vasculature with first pass imaging.

30. The method of claim 27 wherein the injecting step comprises intravenously injecting the contrast agent into an artery, the method further comprising:
performing the MR angiography with at least one selected from the group consisting of steady state imaging, quasi-steady state imaging, or time-delayed imaging.

31. The method of claim 30 wherein the performing step is performed after a build-up of the contrast agent in the patient's bloodstream sufficient to provide a detectable signal for imaging.

32. The method of claim 31 wherein a time for the build-up falls in a range from about 10 minutes to about 2 hours after the injecting step.

33. The method of claim 30 wherein the performing step comprises performing the MR angiography with steady state imaging.

34. The method of claim 30 wherein the performing step comprises performing MR angiography with quasi-steady state imaging.

35. The method of claim 30 wherein the performing step comprises performing the MR angiography with time-delayed imaging.

36. The method of claim 27 further comprising:
performing the MR angiography with a coil tuned for ¹⁹F imaging.

37. The method of claim 27 wherein the contrast agent comprises Gd chelates on its surface, the method further comprising:
performing the MR angiography with a coil tuned for both ¹⁹F and ¹H imaging.

38. The method of claim 27 further comprising:
using spectral peak saturation techniques to reduce signals from unwanted peaks to allow signal localization that avoids chemical shifts.

39. The method of claim 27 further comprising:
using cardiac gating together with sequence optimization to mitigate signal loss during in vivo coronary imaging.

40. The method of claim 8 wherein a field strength for the MR angiography is 1.5 T.

41. The method of claim 40 further comprising:
performing the MR angiography with steady state imaging.

42. The method of claim 41 wherein the MR angiography performing step comprises performing balanced gradient echo imaging.

43. The method of claim 40 wherein the using step comprises using a nontargeted intravascular perfluorocarbon nanoparticle contrast agent emulsion having a dosage in a range from approximately 1.5 to approximately 2.5 mL of emulsion per kg of body weight for an imaging subject.

44. The method of claim 40 further comprising performing the MR angiography with a surface coil.

45. The method of claim 40 further comprising performing the MR angiography with a quadrature birdcage coil.

46. The method of claim 40 further comprising performing the MR angiography with different coils for transmission and reception.

47. The method of claim 8 wherein the nontargeted intravascular perfluorocarbon nanoparticle contrast agent comprises a plurality of cyclic perfluorocarbon molecules, each of the molecules having a plurality of chemically identical fluorine atoms.

48. The method of claim 8 wherein a field strength for the MR angiography is 3 T.

49. The method of claim 8 wherein a field strength for the MR angiography is 7 T.

50. The method of claim 8 wherein the field strength for the MR angiography is greater than 7 T.

51. The method of claim 4 wherein the medical imaging comprises ¹⁹F MR angiography.

52. The method of claim 1 wherein the medical imaging comprises ¹⁹F MR angiography.

53. A method comprising:
using an intravascular fluorinated contrast agent for MR imaging of an interior portion of a body; and
performing spatially matched detection of a plurality of different MR signals to generate contrast agent-enhanced MR images of the interior portion.

54. The method of claim 53 wherein the plurality of different MR signals comprise a ¹⁹F signal and a ¹H signal.

55. The method of claim 53 wherein the contrast agent comprises a plurality of nontargeted intravascular fluorocarbon or perfluorocarbon nanoparticles.

56. The method of claim 55 wherein the plurality of different MR signals comprise a ¹⁹F signal and a ¹H signal.

57. The method of claim 56 wherein the MR imaging comprises MR angiography, and wherein the interior portion comprises a patient's vasculature.

58. The method of claim 57 wherein the MR angiography comprises noninvasive MR angiography.

59. The method of claim 58 wherein the intravascular contrast agent comprises a plurality of perfluorocarbon nanoparticles, each perfluorocarbon nanoparticle having a diameter in a range of about 200 nm to about 300 nm.

60. The method of claim 59 wherein the perfluorocarbon nanoparticles are made by emulsification and are surrounded by a lipid surfactant monolayer.

61. The method of claim 60 wherein the intravascular contrast agent comprises Gd chelates on its surface.

62. The method of claim 61 wherein the intravascular contrast agent remains intravascular while circulating within the bloodstream of the patient.

63. The method of claim 62 wherein the perfluorocarbon nanoparticles are not targeted with any binding ligands.

64. The method of claim 63 wherein the contrast agent comprises a high concentration of fluorine.

65. The method of claim 64 wherein the contrast agent comprises a mixture, the mixture being comprised of approximately 98% perfluorocarbon nanoparticles.

66. The method of claim 63 wherein the perfluorocarbon nanoparticles are liquid at body temperature.

67. The method of claim 63 wherein the MR angiography comprises at least one selected from the group consisting of MR coronary angiography, MR carotid angiography, MR peripheral angiography, MR cerebral angiography, MR arterial, and MR venous angiography.

68. The method of claim 63 further comprising:
interleaving acquisitions from the ¹⁹F signal and the ¹H signal to allow spatial registration of the acquired images.

69. A method comprising:
reducing background tissue signals in MR imaging using ¹⁹F intravascular contrast agents.

70. A method comprising:
using a nontargeted intravascular fluorinated nanoparticle contrast agent for MR imaging of a patient's vasculature;
receiving a ¹⁹F MR signal from the MR imaging;
measuring a difference in the received signal based on a differing concentration of oxygen in the patient's veins and arteries and further based on an effect of relaxation times of ¹⁹F under high and low oxygen tension; and
differentiating venous blood from arterial based at least in part upon the measuring.

71. The method of claim 70 wherein the contrast agent comprises a nontargeted intravascular fluorocarbon or perfluorocarbon nanoparticle contrast agent.

72. The method of claim 70 wherein the MR imaging comprises MR angiography.

73. The method of claim 72 wherein the using step comprises using a nontargeted intravascular perfluorocarbon nanoparticle contrast agent.

74. The method of claim 73 wherein the intravascular perfluorocarbon nanoparticle contrast agent comprises a plurality of perfluorocarbon nanoparticles, each perfluorocarbon nanoparticle having a diameter in a range of about 200 nm to about 300 nm.

75. The method of claim 74 wherein the perfluorocarbon nanoparticles are made by emulsification and are surrounded by a lipid surfactant monolayer.

76. The method of claim 75 wherein the contrast agent remains intravascular while circulating within the bloodstream of the patient.

77. The method of claim 76 wherein the perfluorocarbon nanoparticles are not targeted with any binding ligands.

78. The method of claim 77 wherein the contrast agent comprises a high concentration of fluorine.

79. The method of claim 78 wherein the contrast agent comprises a mixture, the mixture being comprised of approximately 98% perfluorocarbon nanoparticles.

80. A method comprising:
using an intravascular contrast agent for MR imaging of an interior portion of a body, the contrast agent comprising a plurality of nontargeted intravascular fluorinated nanoparticles; and
on the basis of the MR imaging, spectroscopically delineating a concentration of ¹⁹F in a blood pool or vascular space.

81. The method of claim 80 further comprising detecting different ¹⁹F species.

82. The method of claim 81 wherein the contrast agent comprises a plurality of nontargeted intravascular fluorocarbon or perfluorocarbon nanoparticles.

83. The method of claim 82 wherein a plurality of fluorocarbon or perfluorocarbon compounds are used in the nanoparticles, the method further comprising separating different spectral peaks of the plurality of fluorocarbon or perfluorocarbon compounds.

84. A system configured to use a nontargeted intravascular fluorocarbon or perfluorocarbon nanoparticle contrast agent for medical imaging of an interior portion of a body.

85. The system of claim 84 further configured to use the nontargeted intravascular fluorocarbon or perfluorocarbon nanoparticle contrast agent for MR angiography of a patient's vasculature, the MR angiography comprising ¹⁹F MR angiography that is performed via at least one selected from the group consisting of steady state imaging, quasi-steady state imaging, or time-delayed imaging.

86. The system of claim 85 wherein the contrast agent is intravenously injected.

87. A method comprising:
using an intravascular contrast agent for MR imaging of a GI portion of a body, the contrast agent comprising a plurality of nontargeted intravascular fluorinated nanoparticles.

88. The method of claim 87 wherein the contrast agent comprises a plurality of nontargeted intravascular fluorocarbon or perfluorocarbon nanoparticles.

89. A method comprising:
using an intravascular contrast agent for MR cystourethrography, the contrast agent comprising a plurality of nontargeted intravascular fluorinated nanoparticles.

90. The method of claim 89 wherein the contrast agent comprises a plurality of nontargeted intravascular fluorocarbon or perfluorocarbon nanoparticles.
